(19) United States
(12) Patent Application Publication
(10) Pub. No.: US 2012/0289830 A1
(43) Pub. Date: Nov. 15, 2012

(54) METHOD AND ULTRASOUND IMAGING SYSTEM FOR IMAGE-GUIDED PROCEDURES

(75) Inventors: Menachem Halmann, Wauwatosa, WI (US); Michael J. Washburn, Wauwatosa, WI (US)

(73) Assignee: General Electric Company, Schenectady, NY (US)

(21) Appl. No.: 13/104,713

(22) Filed: May 10, 2011

(51) Int. Cl. A61B 8/14

(52) U.S. Cl. 600/443

(57) ABSTRACT

A method and ultrasound imaging system for image-guided procedures includes collecting first position data of an anatomical surface with a 3D position sensor. The method and ultrasound imaging system includes generating a 3D graphical model of the anatomical surface based on the first position data. The method and ultrasound imaging system includes acquiring ultrasound data with a probe in position relative to the anatomical surface. The method and ultrasound imaging system includes using the 3D position sensor to collect second position data of the probe in the position relative to the anatomical surface. The method and ultrasound imaging system includes generating an image based on the ultrasound data and identifying a structure in the image. The method and ultrasound imaging system includes registering the location of the structure to the 3D graphical model based on the first position data and the second position data. The method and ultrasound imaging system includes displaying a representation of the 3D graphical model including a graphical indicator of the structure.

### FIELD OF THE INVENTION

This disclosure relates generally to a method and ultrasound imaging system for generating a representation of a 3D graphical model for use with image-guided procedures.

### BACKGROUND OF THE INVENTION

In many areas, it is typical for a diagnostic imaging system operator to acquire images of a planned site for surgery. Then, a surgeon will use the images in order to plan the most appropriate clinical procedure and approach. Using endocrinology as an example, an endocrinologist will usually acquire images of a patient's neck with an ultrasound imaging system in order to identify one or more lymph nodes that are likely to be cancerous. Next, it is necessary for the endocrinologist to communicate the information regarding the precise location of the one or more cancerous lymph nodes to the surgeon. At a minimum, the endocrinologist needs to identify insertion locations for the surgeon. Preferably, the endocrinologist will also communicate information regarding the depth of various lymph nodes from the skin of the patient, anatomical structures that need to be avoided, the best way to access the lymph node, etc. to the surgeon. However, since a patient may have multiple lymph nodes that need to be involved in the surgical procedure, accurately communicating all the relevant information from the endocrinologist to the surgeon is a difficult and error-prone process.

Therefore, for these and other reasons, an improved method and system for communicating information in image-guided procedures is desired.

### BRIEF DESCRIPTION OF THE INVENTION

The above-mentioned shortcomings, disadvantages and problems are addressed herein which will be understood by reading and understanding the following specification.

In an embodiment, a method for use in an image-guided procedure includes collecting first position data of an anatomical surface with a 3D position sensor and generating a 3D graphical model of the anatomical surface based on the first position data. The method includes acquiring ultrasound data with a probe. The method includes using the 3D position sensor to collect second position data of the probe. The method includes generating an image based on the ultrasound data and identifying a structure in the image. The method includes registering the location of the structure to the 3D graphical model based on the first position data and the second position data. The method also includes displaying a representation of a 3D graphical model including a graphical indicator for the location of the structure.

In another embodiment, a method for use in an image-guided procedure includes collecting first position data by moving a 3D position sensor attached to a probe over an anatomical surface of a patient. The method includes fitting the first position data to a model to generate a 3D graphical model. The method includes identifying a position-of-interest by placing the probe over the position-of-interest and collecting second position data with the attached 3D position sensor. The method includes generating a virtual mark on the 3D graphical model based on the first position data and the second position data. The method includes displaying a representation of the 3D graphical model and the virtual mark, where the location of the virtual mark on the representation of the 3D graphical model corresponds to the location of the position-of-interest with respect to the anatomical surface.

In another embodiment, an ultrasound imaging system includes a probe including an array of transducer elements, a 3D position sensor attached to the probe, a display device, and a processor in electronic communication with the probe, the 3D position sensor, and the display device. The processor is configured to collect first position data from the 3D position sensor while the probe is moved along an anatomical surface. The processor is configured to generate a 3D graphical model based on the first position data. The processor is configured to acquire ultrasound data with the probe. The processor is configured to collect second position data from the 3D position sensor while the probe is acquiring ultrasound data. The processor is configured to generate an image based on the ultrasound data. The processor is configured to register the location of a structure in the image to the 3D graphical model based on the first position data and the second position data. The processor is configured to display a representation of the 3D graphical model on the display device and display a graphical indicator with the representation of the 3D graphical model, wherein the graphical indicator shows the relative positioning of the structure with respect to the anatomical surface.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic diagram of a probe in accordance with an embodiment;

FIG. 3 is a flow chart illustrating a method in accordance with an embodiment; and

FIG. 4 is a schematic representation of a representation of a 3D graphical model in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

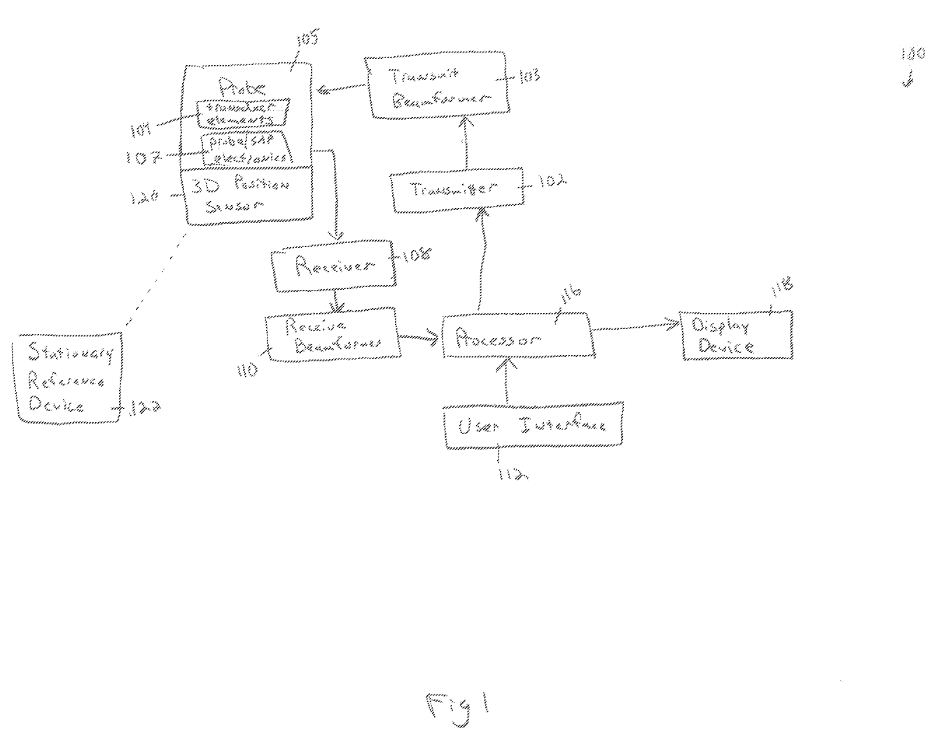

FIG. 1 is a schematic diagram of an ultrasound imaging system 100 in accordance with an embodiment. The ultrasound imaging system 100 includes a transmitter 102 that transmits a signal to a transmit beamformer 103 which in turn drives transducer elements 104 to emit pulsed ultrasonic signals into a structure, such as a patient (not shown). A probe 105 includes the transducer elements 104 and probe/SAP electronics 107. The probe/SAP electronics 107 may be used to control the switching of the transducer elements 104. The probe/SAP electronics 107 may also be used to group the elements 104 into one or more sub-apertures. The transducer elements 104 may be arranged into a variety of geometries. The pulsed ultrasonic signals emitted from the transducer elements 104 are back-scattered from structures in the body to produce echoes that return to the transducer elements 104. The echoes are converted into electrical signals by the transducer elements 104 and the electrical signals are received by a receiver 108. The electrical signals representing the received echoes are passed through a receive beam-former 110 that outputs ultrasound data. For purposes of this disclosure, the term “ultrasound data” may include data that was acquired and/or processed by an ultrasound system. A user interface 112 may be used to control operation of the ultrasound imaging system 100, including, to control the input of patient data, to change a scanning or display parameter, and the like.

The ultrasound imaging system 100 also includes a processor 116 to process the ultrasound data and generate frames or images for display on a display device 118. The processor 116 may be adapted to perform one or more processing operations according to a plurality of selectable ultrasound modalities on the ultrasound data. Other embodiments may use multiple processors to perform various processing tasks. The processor 116 may also be adapted to control the acquisition of ultrasound data with the probe 105. The ultrasound data may be processed in real-time during a scanning session as the echo signals are received. For purposes of this disclosure, the term “real-time” is defined to include a process performed with no intentional lag or delay. An embodiment may update the displayed ultrasound image at a rate of more than 20 times per second. The images may be displayed as part of a live image. For purposes of this disclosure, the term “live image” is defined to include a dynamic image that updates as additional frames of ultrasound data are acquired. For example, ultrasound data may be acquired even as images are being generated based on previously acquired data and while a live image is being displayed. Then, according to an embodiment, as additional ultrasound data are acquired, additional frames or images generated from more-recently acquired ultrasound data are sequentially displayed. Additionally or alternatively, the ultrasound data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the ultrasound signal while a second processor may be used to further process the data prior to displaying an image. It should be appreciated that other embodiments may use a different arrangement of processors.

Still referring to FIG. 1, the ultrasound imaging system 100 may continuously acquire ultrasound data at a frame rate of, for example, 20 Hz to 150 Hz. However, other embodiments may acquire ultrasound data at a different rate. A memory (not shown) may be included for storing processed frames of acquired ultrasound data that are not scheduled to be displayed immediately. In an exemplary embodiment, the memory is of sufficient capacity to store at least several seconds worth of frames of ultrasound data. The frames of ultrasound data are stored in a manner to facilitate retrieval thereof according to the order or time of acquisition. As described hereinabove, the ultrasound data may be retrieved during the generation and display of a live image. The memory may include any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring ultrasound data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well known by those skilled in the art and will therefore not be described in further detail.

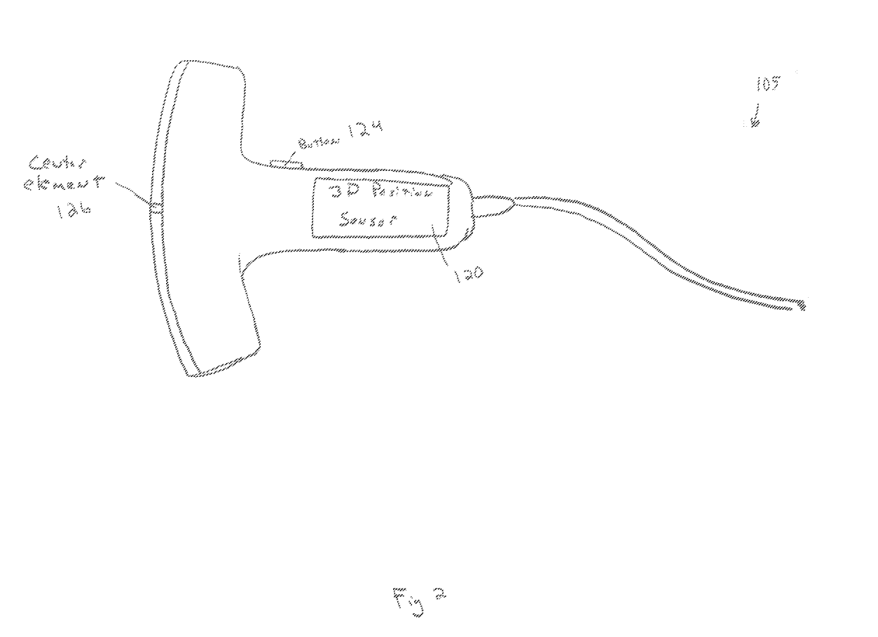

The ultrasound imaging system 100 also includes a 3D position sensor 120 attached to the probe 105. The 3D position sensor 120 may be integral to the probe 105 as shown in FIG. 2, or the 3D position sensor may be attached to the outside of the probe 10 in an easily removable manner (not shown). The 3D position sensor 120 communicates with a stationary reference device 122. Together, the 3D position sensor 120 and the stationary reference device 122 determine position data for the probe 105. In other embodiments, a 3D position sensor may be able to acquire position data without a stationary reference device. The position data may include both position data and orientation data. According to an embodiment, many different samples of position data may be acquired while a sonographer is manipulating the probe 105 and acquiring ultrasound data. The position data may be time stamped, so that it is easily possible to determine the position and orientation of the probe at various times after ultrasound data has been acquired. The 3D position sensor 120 and the stationary reference device 122 may also be used to collect position data of an anatomical surface, as will be discussed in detail hereinafter.

According to an exemplary embodiment, the stationary reference device 122 may be an electromagnetic transmitter, while the 3D position sensor 120 may be an electromagnetic receiver. For example, the electromagnetic transmitter may include one or more coils that may be energized in order to emit an electromagnetic field. The 3D position sensor 120 may likewise include 3 orthogonal coils, such as an x-coil, a y-coil, and a z-coil. The position and orientation of the 3D position sensor 120, and therefore, the probe 105 may be determined by detecting the current induced in each of the 3 orthogonal coils. According to other embodiments, the position of the transmitter and the receiver may be switched so that the transmitter is connected to the probe 105. Electromagnetic sensors are well-known by those skilled in the art and, therefore, will not be described in additional detail.

Additional embodiments may use alternate tracking systems and techniques to determine the position data of the 3D position sensor. For example, a radiofrequency tracking system may be used where a radiofrequency signal generator is used to emit RF signals. Position data is then determined based on the strength of the received RF signal. In another embodiment, an optical tracking system may be used. For example, this may include placing multiple optical tracking devices, such as light-emitting diodes (LEDs) or reflectors on the probe 105 in a fixed orientation. Then, multiple cameras or detectors may be used to triangulated the position and orientation of the LEDs or reflectors, thus establishing the position and orientation of the probe 105. Additional tracking systems may also be envisioned.

In various embodiments of the present invention, ultrasound information may be processed by other or different mode-related modules. A non-limiting list of modes includes: B-mode, Color Doppler, power Doppler, M-mode, spectral Doppler anatomical M-mode, strain, and strain rate. For example, one or more modules may generate B-mode, color Doppler, power Doppler, M-mode, anatomical M-mode, strain, strain rate, spectral Doppler images and combinations thereof, and the like. The images are stored and timing information indicating a time at which the image was acquired in memory may be recorded with each image. The modules may include, for example, a scan conversion module to perform scan conversion operations to convert the image frames from Polar to Cartesian coordinates. A video processor module may be provided that reads the images from a memory and displays the image in real time while a procedure is being carried out on a patient. A video processor module may store the image in an image memory, from which the images are read and displayed. The ultrasound imaging system 100 shown may be configured as a console system, a cart-based system, or a portable system, such as a hand-held or laptop-style system according to various embodiments. The lines shown connecting the components in FIG. 1 may represent physical connections, such as through a cable or wire, or then may represent other types of electronic communication such as including wireless communication. Additionally, the probe 105 may be connected to the processor 116 through an internet or an intranet according to other embodiments.

FIG. 2, is a schematic representation of the probe 105 from the ultrasound imaging system 100 in accordance with an embodiment. The probe 105 is a curved linear probe, but other types of probe may also be used according to other embodiments. Common reference numbers are used to indicate identical structures between FIG. 1 and FIG. 2. FIG. 2 also includes a button 124 and a center element 126 of the transducer array. The functioning of the button 124 and the center element 126 will be discussed hereinafter.

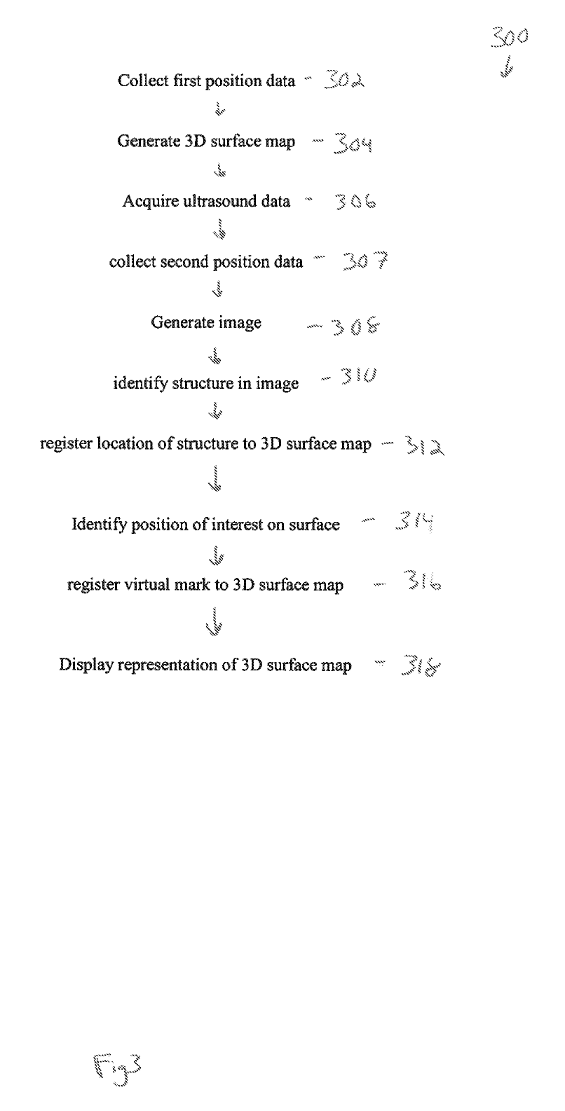

FIG. 3 is a flow chart illustrating a method 300 in accordance with an embodiment. The individual blocks represent steps that may be performed in accordance with the method 300. The technical effect of the method 300 is the display of a representation of a 3D graphical model on a display device such as the display device 118 (shown in FIG. 1). The steps of the method 300 will be described according to an embodiment where the steps are performed with the ultrasound imaging system 100 (shown in FIG. 1). The method 300 will be described according to an exemplary embodiment where a patient's neck is imaged in order to locate the position of one or more lymph nodes for surgical removal. It should be appreciated that the method 300 may be used to identify different structures and/or for different procedures according to other embodiments.

Referring to FIGS. 1, 2, and 3, at step 302, a sonographer collects first position data with the 3D position sensor 120. The sonographer may, for example, move the probe 105 along the surface of a patient's neck. While moving the probe 105 along the patient's neck, the 3D position sensor 120 collects first position data to define at least a portion of the patient's neck surface. The 3D position sensor 120 transmits the first position data to the processor 116. Next, at step 304, the processor 116 generates a 3D graphical model based on the position data. The method 300 may perform differently at step 304 depending upon the quantity and quality of the first position data collected. For example, if the first position data includes a large number of samples, or tracking points, collected over a large enough area of the neck's surface, it may be possible to interpolate the first position data in order to define a surface and generate a 3D graphical model. On the other hand, if the first position data includes a smaller number of samples, it may be advantageous to use a priori information about the structure, in this case a neck, in order to generate the 3D graphical model. For example, it is assumed that the neck is generally cylindrical in shape. Additionally, when using a standard probe, it may be assumed that the sonographer is scanning from the outside surface. As more tracking points are collected, the surface may be updated so as to become more accurate, and less dependent on a priori knowledge. The system may also detect whether the incoming ultrasound information represents real tissue scanning or whether the probe is scanning the air. In the case that the probe is scanning the air, then these 3D tracking points are not representative of the anatomical surface and will not be used for generating the 3D graphical model. In a preferred embodiment a representation of the 3D graphical model will be updated in real time on the ultrasound system's display device and displayed in parallel with a live ultrasound image. The representation of the 3D graphical model may be displayed either side-by-side with the live ultrasound image, or in a top/bottom orientation with the live ultrasound image. According to other embodiments, the 3D graphical model may be displayed as an overlay on top of the live image.

According to other embodiments, the processor 116 may access a deformable model of the intended structure. The deformable model may include multiple assumptions about the shape of the surface. The processor 116 may then fit the first position data to the deformable model in order to generate the 3D graphical model. Any one of the aforementioned techniques may also include the identification of one or more anatomical landmarks to aid in the generation of a 3D graphical model.

Referring to FIGS. 1, 2, and 3, at step 306, the sonographer acquires ultrasound data with the transducer elements 104 in the probe 105. According to an exemplary embodiment, the sonographer may acquire two-dimensional B-mode ultrasound data, but it should be appreciated that other types of ultrasound data may be acquired according to other embodiments including three-dimensional data, one-dimensional data, color data, Doppler data, and M-mode data.

At step 307, the processor collects second position data from the 3D position sensor 120. The second position data may be collected while the ultrasound data is being acquired, or according to other embodiments, the second position data may be collected either before or after the ultrasound data is collected at step 306.

At step 308, the processor 116 generates an image based on the ultrasound data acquired at step 306. The image may optionally be displayed on the display device 118. At step 310, a structure is identified in the image. The structure may be a lymph node in accordance with an exemplary embodiment. The image generated at step 308 may be displayed and the user may identify the position of the structure through a manual process, such as by selecting a region-of-interest including the structure with a mouse or trackball that is part of the user interface 112. According to other embodiments, the processor 116 may automatically identify the structure using an image processing algorithm to detect the shape of the desired structure. As mentioned previously, it may not be necessary to display the image if the processor 116 is used to automatically identify the structure, such as the lymph node. However, according to an embodiment, the user may want to see the image with the automatically identified structure as a way to confirm that the image processing algorithm selected the appropriate structure.

At step 312, the processor 116 registers the location of the structure to the 3D graphical model. Using the second position data, the processor 116 is able to calculate the position and orientation of the probe 105 at the time that the ultrasound data was acquired. The processor 116 is also able to calculate the position of the identified structure within the image generated from the ultrasound data. Therefore, by utilizing the first position data and the second position data, the processor 116 can accurately determine where the structure identified in the image is located with respect to the 3D graphical model.

Still referring to FIGS. 1, 2, and 3, at step 314, the user may identify a position of interest on the anatomical surface. According to an exemplary embodiment, an endocrinologist may be trying to identify the position of one or more lymph nodes that a surgeon will later remove. The endocrinologist may physically mark one or more spots on the anatomical surface corresponding to the locations of suspect lymph nodes. The marks may, for example, indicate insertion locations on the patient's skin that a surgeon could use to access the lymph nodes. According to one work flow, the endocrinologist may place the marks while scanning the patient with the probe 105. Then according to an embodiment, the endocrinologist may place the probe 105 over the marks and actuate a button or switch, such as the button 124 shown in FIG. 2. Each time the user actuates the button 124, the processor 116 stores the position of the probe 105 with respect to the stationary reference device 122 as detected by the 3D position sensor 120. According to another embodiment, the ultrasound imaging system 100 may continuously record position data and the pressing of the button may simply identify the time when the center element 126 is at a specific location. According to other embodiments, the 3D position sensor 107 may be configured so that it captures the data for a different point with respect to the probe 105. For example, the probe 105 may have a small indicator (not shown) or a transparent window (not shown) that the sonographer may place over each of the desired anatomical landmarks before capturing the position data with the 3D position sensor 107. The transparent window may, for example, make it easier for the sonographer to accurately place the probe 105 on a desired anatomical landmark. The user may initiate the storage of the probe's location, and therefore the position of the mark using other user interface devices according to other embodiments, including buttons or switches positioned differently on the probe, buttons or switches located on the user interface 112, and soft keys displayed on the display device 118 and accessed through the user interface 112.

At step 316, the processor 116 registers one or more virtual marks to the 3D graphical model. By correlating the first position data collected by the 3D position sensor at step 302 with the position data collected by the 3D position sensor at step 314, it is relatively easy task for the processor 116 to register the two datasets together in order to define the positions of interest with respect to the anatomical surface.

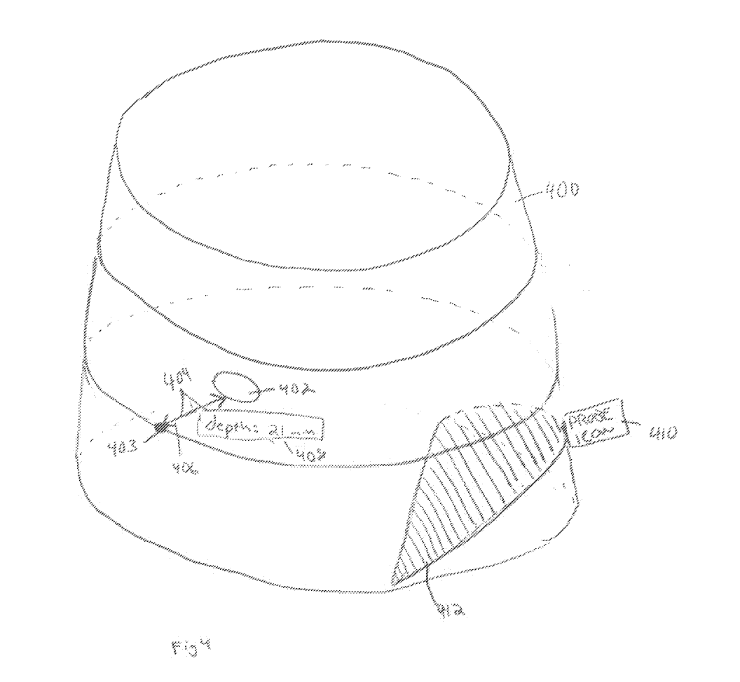

Next, at step 318, the processor 116 displays a representation of the 3D graphical model on the display device 118. FIG. 4 shows an example of a representation of a 3D graphical model 400 in accordance with an embodiment. The representation of the 3D graphical model 400 is of a neck surface. The representation of the 3D graphical model 400 may be similar to volume-rendered images commonly used to display 3D image data according to an embodiment. For example, the representation of the 3D graphical model 400 may be generated through a technique such as ray-casting, which is commonly used to generate volume-rendered images. In typical ray-casting, voxels from an entire volume are all used to generate the final volume-rendered image. However, the 3D graphical model differs from a conventional volume-rendered image because only voxels from the anatomical surface contributes to the representation of the 3D graphical model. The representation of the 3D graphical model 400 captures the geometry of the anatomical surface and may also allow the user to better understand the three-dimensional nature of the surface through the use of visualization techniques such as shading, opacity, color, and the like to give the viewer a better appreciation of depth. According to an embodiment, the user may adjust one or more parameters of the representation of the 3D graphical model 400 in order to focus on a particular region. The user may also use image manipulation techniques including zooming, panning, rotating, and translating of the representation of the 3D graphical model 400 in order to better understand the patient's anatomy.

The representation of the 3D graphical model 400 includes a graphical indicator 402 representing the structure, which may be a lymph node according to an embodiment, and a virtual mark 403. As described previously, the virtual mark 403 may correspond to a particular location of the patient's skin that was identified by the user. According to an embodiment, the location of the virtual mark may have been identified during step 314 of the method 300 (shown in FIG. 3). Additionally, a depth indicator, such as depth indicator 404, may be used to give the user additional information about the position of the structure with respect to the anatomical surface. In FIG. 4, the depth indicator 404 includes both a line 406 and a text box 408. The line 406 indicates the geometrical relationship between the representation of the 3D graphical model 400 and the graphical indicator 402. Additionally, the text box 408, illustrates the depth of the structure beneath the anatomical surface. According to the exemplary embodiment shown in FIG. 4, the lymph node represented by the graphical indicator 402 lies 21 mm beneath the anatomical surface. Other embodiments may use depth indicators of different configurations to illustrate more specific data about the position of the structure or structures indicated by one or more graphical indicators. For example, other embodiments may use a depth indicator including a line with markings at fixed intervals in order to show depth. According to still other embodiments, the depth of the structure may be color-coded based on depth or assigned an opacity based on depth. Any of the these techniques in combination with a 3D surface model helps the user to quickly and accurately determine the positioning of one or more structures with respect to the anatomical surface of the patient. The embodiment shown in FIG. 4 also includes a first icon 410 representing the real-time position of the probe 105 (shown in FIG. 1) and a second icon 412 representing the real-time position of the image being acquired by the probe 105. Both the first icon 410 and the second icon 412 show the position of the probe 105 and the image with respect to the 3D graphical model 400 and help the user to better understand and visualize the relationship between the current ultrasound image and the anatomical surface.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of generating a reference image for use in an image-guided procedure comprising:
collecting first position data of an anatomical surface with a 3D position sensor;
generating a 3D graphical model of the anatomical surface based on the first position data;
acquiring ultrasound data with a probe;
using the 3D position sensor to collect second position data of the probe;
generating an image based on the ultrasound data;
identifying a structure in the image;
registering the location of the structure to the 3D graphical model based on the first position data and the second position data; and
displaying a representation of the 3D graphical model including a graphical indicator of the structure.

2. The method of claim 1, wherein said collecting the first position data occurs while said acquiring ultrasound data with the probe.

3. The method of claim 1, further comprising detecting when the probe is in contact with the anatomical surface and only collecting the first position data from the position sensor while the probe is in contact with the anatomical surface.

4. The method of claim 1, wherein said collecting the second position data of the probe occurs while said acquiring the ultrasound data with the probe.

5. The method of claim 1, further comprising placing a physical mark on the anatomical surface to indicate a location.

6. The method of claim 5, further comprising collecting third position data for the position of the physical mark with the 3D position sensor.

7. The method of claim 6, further comprising displaying a virtual mark on the representation of the 3D graphical model at a location corresponding to the location of the physical mark.

8. The method of claim 7, further comprising displaying a depth indicator associated with the virtual mark.

9. The method of claim 1, further comprising displaying the image based on the ultrasound data at generally the same time as said displaying the representation of the 3D graphical model.

10. The method of claim 1, further comprising displaying a first icon showing the real-time position of the probe with respect to the 3D graphical model.

11. The method of claim 10, further comprising displaying a second icon showing the real-time position of the image with respect to the 3D graphical model.

12. A method for use in an image-guided procedure comprising:
collecting first position data by moving a 3D position sensor attached to a probe over an anatomical surface of a patient;
fitting the first position data to a model to generate a 3D graphical model;
identifying a position-of-interest by placing the probe over the position-of-interest and collecting second position data with the attached 3D position sensor;
generating a virtual mark on the 3D graphical model based on the first position data and the second position data; and
displaying a representation of the 3D graphical model and the virtual mark, where the location of the virtual mark on the representation of the 3D graphical model corresponds to the location of the position-of-interest with respect to the anatomical surface.

13. The method of claim 12, further comprising acquiring ultrasound data with the probe.

14. The method of claim 13, further comprising identifying a structure in an image based on the ultrasound data.

15. The method of 14, further comprising displaying a graphical indicator of the structure on the representation of the 3D graphical model.

16. The method of 12, wherein said identifying the position-of-interest further comprises acquiring the second position data in response to actuating a button or a switch.

17. An ultrasound imaging system for image-guided procedures comprising:
a probe comprising an array of transducer elements;
a 3D position sensor attached to the probe;
a display device; and
a processor in electronic communication with the probe, the 3D position sensor and the display device, wherein the processor is configured to:
collect first position data from the 3D position sensor while the probe is moved along an anatomical surface;
generate a 3D graphical model based on the first position data;
acquire ultrasound data with the probe;
collect second position data from the 3D position sensor while the probe is acquiring the ultrasound data;
generate an image based on the ultrasound data;
register the location of a structure in the image to the 3D graphical model based on the first position data and the second position data;
display a representation of the 3D graphical model on the display device; and
display a graphical indicator with the representation of the 3D graphical model, wherein the graphical indicator shows the relative positioning of the structure with respect to the anatomical surface.

18. The ultrasound imaging system of claim 17, wherein the processor is further configured to display a depth indicator on the representation of the 3D graphical model, wherein the depth indicator illustrates information regarding the depth of the structure with respect to the anatomical surface.

19. The ultrasound imaging system of claim 17, wherein the probe further comprises a button configured to initiate the collection of third position data for a location on the anatomical surface.

20. The ultrasound imaging system of claim 17, wherein the processor is configured to display a volume-rendered image of the 3D graphical model as the representation of the 3D graphical model.

21. The ultrasound imaging system of claim 17, wherein the processor is configured to update the representation of the 3D graphical model in real-time in response to the identification of additional structures either in the image or in an additional image.

22. The ultrasound imaging system of claim 19, wherein the processor is further configured to enable a user to rotate the volume-rendered image of the 3D graphical model on the display device.

23. The ultrasound imaging system of claim 17, wherein the processor is further configured to generate and display the image based on the ultrasound data on the display device in real-time.

24. The ultrasound imaging system of claim 23, wherein the processor is further configured to generate and display the representation of the 3D graphical model on the display device in real-time.
